(19) United States
(12) Patent Application Publication
(10) Pub. No.: US 2026/0355314 A1
(43) Pub. Date: Nov. 12, 2026

(54) RETRO-ACTIVE (DELAYED) IMMUNOLOGICAL TOLERANCE INDUCTION IN PATIENTS WITH PRE-EXISTING SOLID ORGAN TRANSPLANTS

(71) Applicant: The Regents of the University of California, Oakland, CA (US)

(72) Inventors: Jeffrey L. Veale, Los Angeles, CA (US); Nima Nassiri, Los Angeles, CA (US); Neil Kogut, Marina Del Rey, CA (US); Erik Lum, Playa Vista, CA (US)

(73) Assignee: The Regents of the University of California, Oakland, CA (US)

(21) Appl. No.: 19/132,535

(22) PCT Filed: Dec. 18, 2023
(86) PCT No.: PCT/US2023/084521

§ 371 Date: May 23, 2025

Related U.S. Application Data
(60) Provisional application No. 63/433,059, filed on Dec. 16, 2022.
(60) Provisional application No. 63/504,844, filed on May 30, 2023.

(51) Int. Cl. A61K 40/22; A61K 31/517; A61K 35/28; A61K 40/11; A61P 37/06

(52) U.S. Cl. CPC A61K 40/22; A61K 31/517; A61K 35/28; A61K 40/11; A61P 37/06

(57) ABSTRACT

The invention disclosed herein provides methods and materials designed to improve organ transplantation outcomes. Embodiments of the invention include methods designed to provide patients having a pre-existing, functioning solid organ transplant a means of withdrawal from immunosuppressive drugs while maintaining normal function. In embodiments of the invention, this is accomplished through a transfusion of hematopoietic stem/progenitor cells (HSPCs) and T cells from the same donor who provided the original solid organ to the patient, wherein the methods include a conditioning regimen performed on the patient.

### CROSS REFERENCE TO RELATED APPLICATIONS

This application claims the benefit under 35 U.S.C. Section 119(e) of co-pending and commonly-assigned U.S. Provisional Patent Application No. 63/433,059, filed Dec. 16, 2022, and U.S. Provisional Patent Application No. 63/504,844, filed May 30, 2023, all entitled “RETRO-ACTIVE (DELAYED) IMMUNOLOGICAL TOLERANCE INDUCTION IN PATIENTS WITH PRE-EXISTING SOLID ORGAN TRANSPLANTS”, which applications are incorporated by reference herein.

### TECHNICAL FIELD

Embodiments of the disclosure concern at least the fields of medicine, transplantation, immunology and pharmacology.

### BACKGROUND OF THE INVENTION

Advances in surgical techniques and improved drugs that prevent rejection have allowed transplantation of solid organs to become an effective treatment for many diseases. Commonly transplanted organs include heart, intestine, liver, lung, pancreas and kidney.

The major barrier to organ transplantation between genetically non-identical patients lies in the recipient's immune system, which can respond to the transplanted organ as “non-self” and reject it. Thus, having medications to suppress the immune system is essential. However, suppressing an individual's immune system places that individual at greater risk of infections, cancers, heart disease, diabetes, metabolic syndrome, gastrointestinal disorders, neurological disorders, dermatologic and cosmetic disorders, among others. Recipients usually receive a mixture of three maintenance immunosuppressive drugs, including calcineurin inhibitors, antimetabolites, and steroids. Alternative maintenance mediations can include inhibitors of the molecular target of rapamycin (mTOR) and co-stimulation blockade medications. These medications have their own set of detrimental side effects. Ironically, calcineurin inhibitors cause nephrotoxicity from vasoconstriction and ultimately lead to kidney failure, resulting in the need for renal replacement therapy. A leading cause of kidney transplant failure is chronic kidney rejection. Immunosuppressive medications must be continued for the life of the transplanted organ. Even for widely available medications, these costs can be high, which can lead to medication non-adherence and once again, transplant failure.

For the above-noted reasons, the achievement of immunological tolerance is the ultimate aim of transplantation, allowing patients to be liberated from immunosuppression. Operational tolerance is defined as the continued function of the transplanted organ without the need for immunosuppression. It is achieved by introducing hematopoietic progenitor stem cells (HPSCs) from the same solid organ donor to the recipient, after the recipient has been conditioned to receive the HPSCs. Conditioning traditionally includes total lymphoid or full-body irradiation, and an induction agent, such as rabbit antithymocyte globulin (rATG), with or without chemotherapy. Simultaneous tolerance has been demonstrated; for example, the stem cell transplant occurs within a few weeks of kidney transplant while the patient is undergoing induction and conditioning.

However, there are many patients who already have a pre-existing functioning solid organ transplant (e.g., kidney), who will benefit from attaining a tolerance which liberates them from the harmful consequences of drug mediated immunosuppression. Such tolerance will allow a patient's transplanted organ to potentially function for life.

The invention disclosed herein meets needs in this technology by providing regimens that can generate (delayed) immunological tolerance and successful durable chimerism in transplant patients.

### SUMMARY OF THE INVENTION

Embodiments of the invention are designed to address problems observed in immunosuppression regimens used in organ transplant recipients, and include, for example, methods of modulating chimerism and/or immunological tolerance in a patient having a pre-existing transplanted organ (e.g., a transplanted kidney, heart, intestine, liver, pancreas or lung). The invention disclosed herein includes therapeutic methods designed so that patients with a pre-existing, well-functioning kidney transplant from a living donor can be retroactively withdrawn from immunosuppressive medications without compromising allograft function. As discussed below, this can be achieved through infusions of hematopoietic progenitor stem cells (CD34⁺) and T-cells (CD3⁺) infusions from the same/original solid organ donor. These cells can, for example, be mobilized from the same/original transplant donor using a combination of granulocyte colony stimulating factor (gCSF) and plerixafor. After a cell collection procedure such as apheresis, the collected hematopoietic progenitor stem cells (CD34⁺) and T-cells (CD3⁺) cells can be processed, separated, and evaluated for sterility and quality. In typical embodiments of the invention, hematopoietic progenitor stem cells (CD34⁺) and T-cells (CD3⁺) infusion in the patient is preceded by a conditioning regimen of total lymphoid irradiation (TLI) and rabbit anti-thymocyte globulin (rATG), thereby avoiding the need for chemotherapy. As discussed below, embodiments of the invention have been discovered to achieve durable mixed chimerism.

On Feb. 24, 2023, UCLA performed the first ever retroactive tolerance 417 days after kidney transplantation. Durable stem cell engraftment and mixed chimerism was demonstrated at six and ten weeks, with 28% and 46% donor stem cells. Previous experience with simultaneous tolerance demonstrates that when this level of engraftment and durable chimerism is achieved, withdrawal of immunosuppression is inevitable. This inaugural retroactive tolerance patient has already been removed from two of the three maintenance immunosuppressive medications, and is only on low-dose tacrolimus, which will be continually weaned. Illustrative patient chimerism results are shown in Table 1 below.

In embodiments of the invention, as part of delayed tolerance monitoring steps, graft function and chimerism can be evaluated in recipient whole blood and white blood cell subsets. As therapeutic milestones are then met in the recipient, immunosuppressive medications can be weaned/discontinued. For example, weaning of immunosuppressive agents such as calcineurin inhibitors can begin at 6 months with a goal of discontinuation by 12 months as long as the following conditions are met: (1) chimerism is detectable for at least 180 days after CD34⁺ and CD3⁺ cell infusion, (2) stable graft function (defined as eGFR >30 mL/min and no greater than sustained 30% change over 3 months from baseline function) without clinical rejection episodes is maintained, and (3) there is no evidence of graft versus host disease (GVHD).

The invention disclosed herein includes methods that provide patients living with a transplanted organ (e.g., a transplanted kidney, heart, intestine, liver, pancreas or lung) a means of delayed withdrawal from immunosuppressive drugs while maintaining normal organ function. In this way, embodiments of the invention include methods of modulating immunological tolerance in a patient having a transplanted organ or the like. Typically, these methods comprise selecting a patient having a transplanted organ, wherein the transplanted organ is from an individual who is not genetically identical to the patient, and the patient is administrated an immunosuppressive agent to facilitate immunological tolerance of the transplanted organ. These methods then comprise performing a transplantation conditioning regimen on the patient, wherein the transplantation conditioning regimen comprises the administration of rabbit anti-thymocyte globulin and total lymphoid irradiation, optionally with the administration of belumosudil (depending on HLA and eplet matching); and then further transfusing the patient with hematopoietic stem/progenitor cells; wherein the hematopoietic stem/progenitor cells are obtained from the same individual from which the transplanted organ was obtained. In this way, immunological tolerance in the patient having a transplanted organ is modulated.

Illustrative specific embodiments of the invention include methods that provide patients having a pre-existing, well-functioning kidney transplant a means of withdrawal from immunosuppressive drugs while maintaining normal renal function. This is accomplished through a transfusion of hematopoietic stem/progenitor cells (HSPCs) from the same donor who provided the original kidney transplant. Donor HSPCs are peripheralized using granulocyte colony stimulating factor and plerixafor, after which cells are collected, processed, and evaluated. Recipients undergo a conditioning regimen of total lymphoid irradiation (TLI) and rabbit anti-thymocyte globulin (rATG) optionally with the administration of belumosudil (depending on HLA and eplet matching), after which a stem cell infusion is performed. In embodiments of the invention, immunosuppression can be discontinued within 12 months after HSPC transfusion if the following conditions are met: (1) chimerism is detectable for at least 180 days after CD34⁺ and CD3⁺ cell infusion, (2) stable graft function (defined as eGFR >30 mL/min and no greater than sustained 30% change over 3 months) without clinical rejection episodes is maintained, (3) there is lack of rejection and (4) there is no evidence of graft vs. host disease. The purpose is to maximize allograft function, minimize comorbidities associated with immunosuppression, and minimize the need for re-transplantation.

As noted above, embodiments of the invention include observing a physiological parameter in the patient for a period of time following transfusing the patient with hematopoietic stem/progenitor cells (e.g., for at least 3, 6, 9 or 12 months). Illustrative physiological parameters includes at least one of: detectability of chimerism; estimated glomerular filtration rate (eGFR); and the presence of graft versus host disease. Embodiments of the invention can also include the step of withdrawing the administration of the immunosuppressive agent when the patient exhibits an estimated glomerular filtration rate >30 mL/min and no greater than a 30% change in estimated glomerular filtration rate for at least 3 months. In certain embodiments of the invention, the hematopoietic stem/progenitor cells are obtained by administering one or more mobilization agents to the donor, wherein the one or more mobilization agents comprise plerixafor, human granulocyte colony-stimulating factor (G-CSF), or a combination thereof. In various embodiments of the invention, the immunosuppressive agent can comprise various immunosuppressive agents. Optionally, the transplanted cells comprise at least 1, 2, 3, 4, 5 or 6 human leukocyte antigens that are mismatched with human leukocyte antigens present in the patient/recipient. In certain embodiments of the invention, the HLA mismatches occur at least one of: HLA-A, HLA-B, HLA-C or HLA-DR. In some embodiments of the invention, the donor is HLA-identical.

The methods of the invention can be used in a variety of transplantation procedures such as kidney transplantation. In illustrative embodiments of the invention, the transplanted tissues or organs comprise at least one of: kidney cells, heart cells, intestinal cells, liver cells, lung cells or pancreatic cells. In some embodiments of the invention, the tissues or organs are obtained from a human lymphocyte antigen (HLA) single haplotype-matched donor. In other embodiments of the invention, the one or more genetically non-identical cells, tissues or organs are obtained from a 0-3 antigen HLA mismatched donor (e.g., 0-3 HLA mismatches occurring at least one of: HLA-A, HLA-B, HLA-C or HLA-DR).

Freeing patients from immunosuppression regimens will eliminate the toxicities associated with immunosuppression, decrease the risks for short and long-term infectious and cancerous complications, reduce or eliminate the risk of graft rejection, and increase allograft durability, potentially for life. By optimizing allograft survivability, the burden of re-transplantation is minimized, and challenges to the patient and the health care system as a result of graft failure/rejection and repeat renal replacement are mitigated. Methods that allow for increasing kidney allograft life-years consequently allows available kidneys to be allocated elsewhere; with kidneys that would be allocated to patients with graft failure instead being directed towards the transplant-naïve patients. With such methods, chains of transplantations will be more efficacious and patients can move more rapidly up the deceased donation waiting list. In summary, the potential impact on increasing the pool of organ donors increases substantially with any advancements in immunological tolerance.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1: Cartoon schematic showing a retroactive tolerance protocol (adapted from the Stanford simultaneous tolerance protocol). Months to years after the initial transplant event, the donor's stem cells are mobilized using a combination of granulocyte colony stimulating factor and plerixafor. The cells are separated, processed, and cryopreserved. The recipient, who has already recovered from the kidney transplant, undergoes conditioning with rabbit antithymocyte globulin and total lymphoid irradiation, after which the donor-derived stem cells are infused. Serial chimerism testing guides weaning of immunosuppression, with eventual full withdrawal within one year after stem cell infusion.

### DETAILED DESCRIPTION OF THE INVENTION

In the description of embodiments, reference may be made by way of illustrative embodiments by which the invention may be practiced. It is to be understood that other embodiments may be utilized, and structural changes may be made without departing from the scope of the present invention.

Approximately 20,000 kidney transplants are performed in the United States annually. Although kidney transplantation prolongs survival and improves the quality of life of patients with end-stage renal disease, it has two major limitations: the risk of graft rejection and the need for lifelong immunosuppression. Conventional immunosuppression following kidney transplantation is associated with serious, long-term complications, including infection, secondary malignancies, hypertension, hyperlipidemia, diabetes, and cardiovascular disease. Calcineurin inhibitors may damage kidney function and contribute to graft failure. In addition, the intensive and complicated pharmaceutical regimen adversely affects quality of life and leads to a risk of medication non-adherence. Moreover, the costs of conventional immunosuppression are high, and ironically contribute to long-term graft failure.

In spite of immunosuppression regimens, most kidney grafts eventually fail. According to recent data from the Organ Procurement and Transplant Network, the graft half-life following living donor transplantation treated with conventional immunosuppression is approximately 12-15 years. Following graft failure, patients must return to dialysis and/or undergo re-transplantation.

Embodiments of the invention are designed to address problems observed in immunosuppression regimens, and include, for example, methods of modulating chimerism and/or immunological tolerance in a patient having a pre-existing transplanted organ (e.g. a transplanted kidney, heart, intestine, liver, pancreas or lung). With the twin aims of prolonging graft survival and eliminating (or at least minimizing) immunosuppression, investigators in the field of transplantation have long sought to find a reliable and safe method to induce transplant tolerance (defined as a state of immunological non-responsiveness to a transplanted organ in the absence of immunosuppression). When transplanting tissues, medical practitioners consider blood type and human leukocyte antigen (HLA) matching in order to optimize the chances of a successful transplantation. Retroactive (delayed) tolerance is applicable both in perfect HLA and imperfect HLA matching. Molecular matching (eplet) can be utilized. For example, in some embodiments of the invention, the one or more genetically non-identical cells, tissues or organs comprise at least one human leukocyte antigen that is mismatched with a human leukocyte antigen present in the patient.

Typically these methods include the steps of first selecting a patient having a transplanted organ, wherein the transplanted organ is from an individual who is not genetically identical to the patient and the patent is administrated an immunosuppressive agent to facilitate immunological tolerance of the transplanted organ; and then performing a transplantation conditioning regimen on the patient, wherein the transplantation conditioning regimen comprises the administration of rabbit anti-thymocyte globulin and total lymphoid irradiation; and further transfusing the patient with donor hematopoietic stem/progenitor cells and T cells; wherein the donor hematopoietic stem/progenitor and T cells are obtained from the individual from which the transplanted organ was obtained. In certain embodiments of the invention, the transplanted organ/cells comprise at least 1, 2, 3, 4, 5 or 6 human leukocyte antigens that are mismatched with human leukocyte antigens present in the patient. Typically, the HLA mismatches occur at least one of: HLA-A, HLA-B, HLA-C or HLA-DR. In some embodiments of the invention, the donor is HLA-identical. In certain embodiments of the invention, the hematopoietic stem/progenitor cells used in the methods are obtained by administering one or more mobilization agents to the donor, wherein the one or more mobilization agents comprise plerixafor, human granulocyte colony-stimulating factor (G-CSF), or a combination thereof.

Embodiments of the invention can include the step of further observing a physiological parameter in the patient for a period of time following transfusing the patient with hematopoietic stem/progenitor cells, wherein the physiological parameter includes at least one of: detectability of chimerism; estimated glomerular filtration rate (eGFR); and the presence of graft versus host disease. Typically, in these embodiments, the period of time is at least 3, 6, 9 or 12 months. Embodiments of the invention can further include the step then withdrawing the administration of the immunosuppressive agent when the patient exhibits a selected physiological profile such as an estimated glomerular filtration rate >30 mL/min and no greater than a 30% change in estimated glomerular filtration rate for at least 3 months. In certain embodiments of the invention, the immunosuppressive agent comprises an antibody (e.g., Daclizumab and Basiliximab), a calcineurin inhibitor (e.g. cyclosporine, tacrolimus), an antimetabolite (e.g. mycophenolate mofetil, azathioprine), an mTORi (e.g. sirolimus, everolimus), and/or a steroid (e.g. prednisone).

Certain embodiments of the invention focus on patients transplanted with an organ less than 30 days (e.g., less than 3, 2 or 1 week(s)) prior to performing a transplantation conditioning regimen on the patient and then transfusing the patient with hematopoietic stem/progenitor cells and T cells. Other embodiments of the invention focus on patients transplanted with an organ more than 30 days prior to performing a transplantation conditioning regimen on the patient and then transfusing the patient with hematopoietic stem/progenitor cells and T cells. For example, some embodiments of the invention focus on patients transplanted with an organ more than 1 year, 2 years, 3 years, 4 years or 5 years prior to performing a transplantation conditioning regimen on the patient and then transfusing the patient with hematopoietic stem/progenitor cells and T cells obtained from the organ donor.

Chimerism analysis is a well-established method for monitoring the state of hematopoietic stem cell transplantation (HSCT) over time by analyzing peripheral blood or bone marrow samples of the recipient. See, e.g., Tozzo et al., Diagnostics (Basel). 2021 April; 11(4): 621. Embodiments of the invention include chimerism analyses. For example, certain embodiments of the invention observed chimerism detectable in the patient for at least 180 days after hematopoietic stem/progenitor cells and T cell infusion. In certain of these methods, the patient exhibits a chimerism of at least 5%, 10%, 20% or 40% following the hematopoietic stem/progenitor cells and T cell infusion. Typically in these methods, chimerism is observed when donor leukocytes comprise at least 5%, 10%, 20% or 40% of peripheral blood leukocytes obtained from the patient.

Embodiments of the invention can further include the step of weaning a patient off of the immunosuppressive agent when at least one of the following conditions are met: (1) chimerism is detectable for at least 180 days after hematopoietic stem/progenitor cells and T cell transfusion, (2) stable graft function (defined as eGFR >30 mL/min and no greater than sustained 30% change over 3 months from baseline function) without clinical rejection episodes is maintained, and (3) there is no evidence of graft versus host disease (GVHD). In certain embodiments, weaning the patient off of the immunosuppressive agent occurs over at least 20, 30, 40 or 50 weeks.

The Rock-2 inhibitor belumosudil (KD025) demonstrates several properties which make it an attractive agent for use in certain embodiments of the invention. Embodiments of the invention include methods for administering the drug belumosudil to patients who have undergone tissue and/or organ transplantation. Typically, these methods for administering belumosudil comprise the steps of selecting a patient for belumosudil administration, wherein the patent is selected as one having undergone a transplantation procedure with one or more genetically non-identical cells, tissues or organs; and then administering belumosudil to the patient. Typically, in these methods, the administration of belumosudil to the patient occurs in conjunction with a transplantation conditioning regimen comprising anti-thymocyte globulin (rATG) administration and total lymphoid irradiation (TLI).

Belumosudil increases regulatory T-cell number and function and shifts the balance of effector/regulatory T cells towards the latter. Belumosudil reduces IL-17 and IL-21 through reduction in stat 3 phosphorylation. These inflammatory cytokines may play a role in graft rejection. Belumosudil down-regulates key fibrotic processes in preclinical models, including profibrotic gene transcription, stress fiber formation, myofibroblast activation and collagen deposition. Interstitial fibrosis is a prominent histological feature of chronic graft rejection. Belumosudil has been given a breakthrough drug designation by the FDA for the treatment of chronic graft versus host disease (GVHD). A recent report of a phase 2 trial showed a high response rate in advanced, heavily pre-treated chronic GVHD. For further discussions of belumosudil, see, e.g., PCT Publications: WO2006105081; WO2008054599; WO2010104851; WO2014055996 US Patent Publication Nos. 20170112832, 20160237095 and 20150238601; and also Boerma et al., Blood Coagul Fibrinolysis, 2008, 19(7):709-718, the contents of each of which is incorporated herein by reference.

Embodiments of the present invention utilize pharmaceutically acceptable compositions which comprise a therapeutically-effective amount of belumosudil formulated together with one or more pharmaceutically acceptable carriers (additives) and/or diluents. The phrase “therapeutically-effective amount” as used herein means that amount of a compound, material, or composition comprising belumosudil of the present invention which is effective for producing some desired therapeutic effect in at least a sub-population of cells in a patient at a reasonable benefit/risk ratio applicable to any medical treatment, e.g. reasonable side effects applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals with toxicity, irritation, allergic response, or other problems or complications, commensurate with a reasonable benefit/risk ratio. The phrase “pharmaceutically-acceptable carrier” as used herein means a pharmaceutically-acceptable material, composition or vehicle, such as a liquid or solid filler, diluent, excipient, manufacturing aid (e.g., lubricant, talc, magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient.

Actual dosage levels of belumosudil in the pharmaceutical compositions of this invention may be varied so as to obtain an amount of the active ingredient which is effective to achieve the desired therapeutic response for a particular patient, composition, and mode of administration, without being toxic to the patient. The selected dosage level can depend upon a variety of factors including the activity of the particular compound of the present invention employed, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts. Using the disclosure provided herein, a physician having ordinary skill in the art can readily determine and prescribe the effective amount of the pharmaceutical belumosudil composition used (e.g., 200 mg QD, 200 mg BID, and 400 mg QD). For example, the physician or provider could start doses of the compounds of the invention employed in the pharmaceutical composition at levels lower than that required in order to achieve the desired therapeutic effect and gradually increase the dosage until the desired effect is achieved.

In certain embodiments, a dose of a compound or a composition is administered to a subject every day, every other day, every couple of days, every third day, once a week, twice a week, three times a week, or once every two weeks. If desired, the effective daily dose of the active compound may be administered as two, three, four, five, six or more sub-doses administered separately at appropriate intervals throughout the day, optionally, in unit dosage forms. In some embodiments, a dose(s) of a compound or a composition is administered for 2 days, 3 days, 5 days, 7 days, 14 days, or 21 days. In certain embodiments, a dose of a compound or a composition is administered for 1 month, 1.5 months, 2 months, 2.5 months, 3 months, 4 months, 5 months, 6 months or more.

The methods of the invention can be used in a variety of transplantation procedures, including but not limited to kidney transplantation. In illustrative embodiments of the invention, the one or more genetically non-identical cells, tissues or organs comprises at least one of: kidney cells, heart cells, intestinal cells, liver cells, lung cells or pancreatic cells. Moreover, certain embodiments of the invention are used in vascular composite allografts. Vascular composite allografts involve the transplantation of multiple structures that may include skin, bone, muscles, blood vessels, nerves and connective tissue. Face, uterine, and hand transplants are the most well-known types of vascular composite allografts. The use of vascular composite allografts transplantation is increasing as this technology matures and further includes, for example, the transplantation of reproductive organs. Retroactive tolerance may be applicable in this setting as well.

### EXAMPLES

### Example 1: Retro-Active Immunological Tolerance after Kidney Transplantation

### Summary

Immunological tolerance based on the Stanford University Protocol combines simultaneous hematopoietic stem cells with kidney transplantation in a prospective fashion to liberate patients from immunosuppression. However, patients who have been transplanted in the past also desire the opportunity to be freed from the side effects of anti-rejection medications. Herein, we report the first-ever case of delayed tolerance in a patient with a pre-existing well-functioning kidney transplant. Donor stem cells were infused into the recipient 417 days following kidney transplantation. This resulted in successful engraftment, without producing a profound antibody mediated response or rejection of the recipient's renal transplant once the donor antigens were reintroduced. Durable chimerism was achieved and all immunosuppression medications were tapered-off. The patient remains tolerant and has exhibited his best renal allograft function after all immunosuppression was removed. Of note, the conditioning and stem cell infusion were completely performed in the outpatient setting.

### Brief Report

A recent New York Times article highlights the devastating burdens of chronic immunosuppression after organ transplantation and describes the “gratitude paradox,” namely that the appreciation of the gift of an organ transplant simultaneously discourages transplant recipients from talking freely about the real problems they face and the compromising and life-threatening side effects of the medicines they must take.¹ Current immunological tolerance protocols are limited by design to transplant naïve patients eligible for de novo combined solid organ and hematopoietic progenitor stem cell transplantation²⁻⁸. To be truly impactful, the pool of eligible candidates for tolerance must be expanded to include recipients living with a pre-existing solid organ transplant who's donor is still alive.

### Case Presentation

A 28 year-old male with end-stage renal disease on hemodialysis from biopsy-proven IgA nephropathy underwent a HLA matched living-related renal transplant from his brother. His post-operative course was unremarkable, and he was maintained using standard immunosuppression medications.

Due to the recipient's young age and concern over decades of immunosuppression exposure, he was consented and enrolled in an RB-approved (#22-001470) delayed (retroactive) tolerance trial at our center (NCT05525507).

Fourteen months following the kidney transplant, the same living-related kidney donor was administered granulocyte colony stimulating factor and plerixafor for stem cell mobilization. Donor apheresis was followed by cell separation and processing. CD34+ and CD3+ cells were cryopreserved in preparation for infusion into the recipient.

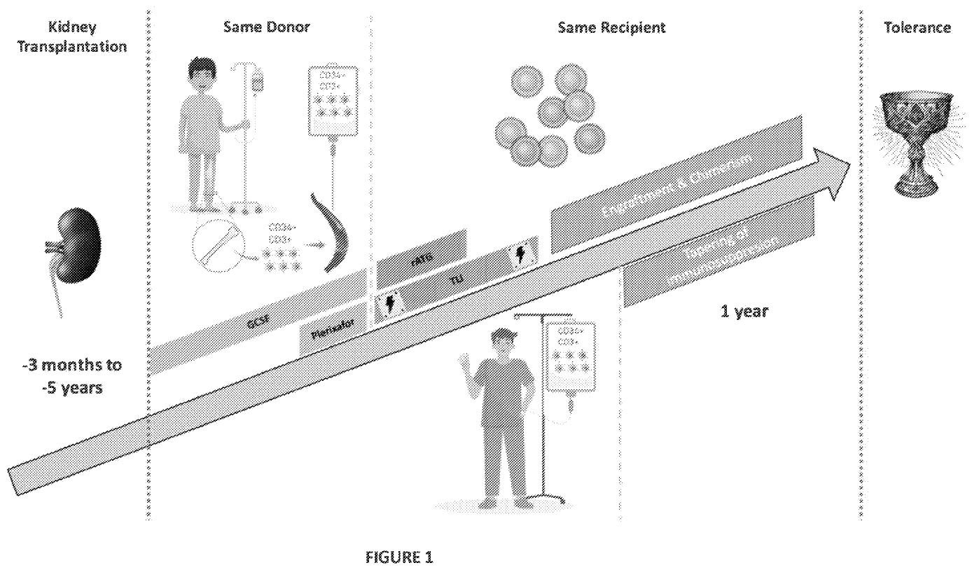

The recipient underwent a conditioning regimen of rabbit anti-thymocycte globulin and total lymphoid irradiation (FIG. 1). After conditioning, the donor's cryopreserved stem cells were thawed and infused through a peripherally-inserted central catheter. The recipient received immunosuppression as depicted in FIG. 1. All components of this delayed tolerance protocol were completed on an outpatient basis.

Four weeks following stem cell infusion the recipient's serum creatinine rose from a baseline of 1.4 mg/dL to 1.9 mg/dL, prompting a biopsy. There was no evidence of rejection or recurrent IgA nephropathy. Interestingly, the biopsy demonstrated evidence of chronic calcineurin-inhibitor toxicity related to tacrolimus exposure prior to tolerance conditioning.

Post stem cell transplantation, laboratory testing demonstrated durable chimerism (Table 2) and he was successfully weaned off all immunosuppression medication on day 228 post stem cell transplant. His creatinine remained elevated between 1.8-2.4 mg/dL until tacrolimus weaning was initiated, following which his creatinine gradually decreased. Upon complete withdrawal of tacrolimus his renal allograft achieved its best function, with a serum creatinine of 1.2 mg/dL and testing showed persistent low level chimerism.

### DISCUSSION

This is the first reported case of immune tolerance asynchronously after initial kidney transplantation. Up until now immunological tolerance has only been realized prospectively in transplant-naïve patients. Yet, many more patients have well-functioning kidney transplants than those who are currently eligible for simultaneous stem cell and kidney transplant. Assuming that their donors are alive and willing, these patients may now have the opportunity to be liberated from immunosuppression for those who have already received a kidney transplant years in the past.

A major concern with this delayed tolerance approach is the administration of donor-derived stem cells is that it reintroduces donor antigens. This sensitizing event can result in a profound recipient response, one that may trigger rejection and even the devastating loss of the living donor transplant. This process did not occur in our case. Successful engraftment of stem cells and durable chimerism was achieved with rabbit anti-thymocyte globulin and total lymphoid irradiation, allowing for tolerance and complete removal of maintenance immunosuppression. A second patient has undergone our protocol and has also exhibited successful engraftment and durable chimerism, and is currently in the process of tapering immunosuppression.

The delayed tolerance approach has several benefits for transplant recipients. As seen in this case, withdrawal of tacrolimus results in improvement in renal allograft function. Calcineurin inhibitors are known nephrotoxins which result in progressive allograft deterioration and return to dialysis. Achieving tolerance and withdrawal of immunosuppression can increase the life of a transplant, preventing the need for re-transplantation and permitting one transplant for life.

Delayed tolerance may allow for tolerance trials to other solid organ transplant recipients. Heart, lung, and liver transplant recipients are often critically ill at the time of transplantation and may not be suitable for conditioning therapy immediately following organ transplantation. A delayed tolerance approach would permit adequate time for recovery and rehabilitation prior to undergoing the condition regimen.

All donor and recipient components of this protocol were conducted in an outpatient setting. Currently, conditioning regimens for simultaneous tolerance require prolonged admission to the hospital. Performing tolerance in the outpatient setting takes the strain off of the operating rooms, medical staff, hospital bed capacity, and is much more cost effective.

Following transplantation, patient's face a “gratitude paradox,” which limits their ability to discuss the toxic side effects of immunosuppression for fear of appearing ungrateful for their gift¹. This inaugural delayed tolerance case provides evidence that patients who have already been transplanted may be included in future tolerance trials and potentially freed from the burdens of chronic immunosuppression.

### REFERENCES

1. Opinion|My Transplanted Heart and I Will Die Soon—The New York Times. https://www.nytimes.com/2023/04/18/opinion/heart-transplant-donor.html. Accessed May 16, 2023.
2. Sykes M, Chandran S, Kawai T, et al. Meeting Report: The Fifth International Samuel Strober Workshop on Clinical Immune Tolerance. Transplantation. 2023; 107(3):564-569. doi:10.1097/TP.0000000000004473
3. Nassiri N, Lum E, Mead M D, Raldow A C, Kogut N, Veale J L. Immune tolerance induction through haematopoietic chimerism after kidney donation. Lancet (London, England). 2022; 400(10350):e2. doi:10.1016/S0140-6736(22)00914-X
4. Leventhal J, Abecassis M, Miller J, et al. Tolerance induction in HLA disparate living donor kidney transplantation by donor stem cell infusion: Durable chimerism predicts outcome. Transplantation. 2013; 95(1):169-176. doi:10.1097/TP.0b013e3182782fc1
5. Scandling J D, Busque S, Dejbakhsh-Jones S, et al. Tolerance and withdrawal of immunosuppressive drugs in patients given kidney and hematopoietic cell transplants. Am J Transplant. 2012; 12(5):1133-1145. doi:10.1111/J.1600-6143.2012.03992.X
6. Kawai T, Sachs D H, Sprangers B, et al. Long-term results in recipients of combined HLA-mismatched kidney and bone marrow transplantation without maintenance immunosuppression. Am J Transplant. 2014; 14(7):1599-1611. doi:10.1111/ajt.12731
7. Fehr T, Hübel K, de Rougemont O, et al. Successful Induction of Specific Immunological Tolerance by Combined Kidney and Hematopoietic Stem Cell Transplantation in HLA-Identical Siblings. Front Immunol. 2022; 13. doi:10.3389/FIMMU.2022.796456
8. Yeshurun M, Rachamimov R, ShargianL. Combined Kidney and Hematopoietic Cell Transplantation for Tolerance Induction between HLA Matched Sibling Donor-Recipient Pairs. Transpl Cell Ther. 2021; 29(2S):S1-S468.
9. Scandling J D, Busque S, Dejbakhsh-Jones S, et al. Tolerance and chimerism after renal and hematopoietic-cell transplantation. N Engl J Med. 2008; 358(4):362-368. doi:10.1056/NEJMOA074191

All publications mentioned herein (e.g., Busque, S., et al., Mixed chimerism and acceptance of kidney transplants after immunosuppressive drug withdrawal. Sci Transl Med, 2020. 12(528); Scandling, J. D., et al., Chimerism, graft survival, and withdrawal of immunosuppressive drugs in HLA matched and mismatched patients after living donor kidney and hematopoietic cell transplantation. Am J Transplant, 2015. 15(3): p. 695-704; Ferreira, L. M. R., et al., Next-generation regulatory T cell therapy. Nat Rev Drug Discov, 2019. 18(10): p. 749-769; Cutler C S., et al., Belumosudil for Chronic Graft-versus-Host Disease (cGVHD) After 2 or More Prior Lines of Therapy: The ROCKstar Study. Blood. 2021 Jul. 15:blood.2021012021. doi: 10.1182/blood.2021012021; Jagasia et al., ROCK2 Inhibition With Belumosudil (KD025) for the Treatment of Chronic Graft-Versus-Host Disease. J Clin Oncol. 2021 Jun. 10; 39(17):1888-1898. doi: 10.1200/JCO.20.02754. Epub 2021 Apr. 20. PMID: 33877856; PMCID: PMC8189612; and Issa F., et al The Fourth International Workshop on Clinical Transplant Tolerance. Am J Transplant. 2021 Jan. 21(1):21-31. doi: 10.1111/ajt.16139) are incorporated by reference to disclose and describe aspects, methods and/or materials in connection with the cited publications. Many of the techniques and procedures described or referenced herein are well understood and commonly employed by those skilled in the art. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

### TABLES

TABLE 1
Chimerism Testing Report
Donor P101D, Date of Bone Marrow Transplant: Feb. 24, 2023

| Sample | Received | Sample | Specimen | | Days Post | Donor | Recipient |
|---|---|---|---|---|---|---|---|
| Date | Date | ID | Source | Cell Type | Transplant | Cells | Cells |
| Apr. 19, 2023 | Apr. 19, 2023 | 789252 | PB | Unfractionated | 54 | 43% | 57% |
| | | | | PBMC | | | |
| Apr. 19, 2023 | Apr. 19, 2023 | 789252 | PB CD3+ | CD3 | 54 | 4% | 96% |
| Apr. 19, 2023 | Apr. 19, 2023 | 789252 | CD19+ | CD19 | 54 | 63% | 37% |
| Apr. 19, 2023 | Apr. 19, 2023 | 789252 | CD56+ | CD56 | 54 | 17% | 83% |
| Apr. 19, 2023 | Apr. 19, 2023 | 789252 | CD33+ CD66+ | CD33 | 54 | 46% | 54% |
| Mar. 20, 2023 | Mar. 20, 2023 | 788049 | PB | Unfractionated | 24 | 26% | 74% |
| | | | | PBMC | | | |
| Mar. 20, 2023 | Mar. 20, 2023 | 788049 | PB CD3+ | CD3 | 24 | 2% | 98% |
| Mar. 20, 2023 | Mar. 20, 2023 | 788049 | CD19+ | CD19 | 24 | 21% | 79% |
| Mar. 20, 2023 | Mar. 20, 2023 | 788049 | CD56+ | CD56 | 24 | 3% | 97% |
| Mar. 20, 2023 | Mar. 20, 2023 | 788049 | CD33+ CD66+ | CD33 | 24 | 28% | 72% |
| Jan. 6, 2023 | Jan. 6, 2023 | 785414 | PB | Unfractionated | 368 | 1%% | 99% |
| | | | | PBMC | | | |
| Jan. 6, 2023 | Jan. 6, 2023 | 785414 | PB CD3+ | CD3 | 368 | 1% | 99% |
| Jan. 6, 2023 | Jan. 6, 2023 | 785414 | CD19+ | CD19 | 368 | 2% | 98% |
| Jan. 6, 2023 | Jan. 6, 2023 | 785414 | CD56+ | CD56 | 368 | 2% | 98% |
| Jan. 6, 2023 | Jan. 6, 2023 | 785414 | CD33+ CD66+ | CD33 | 368 | 2% | 98% |

This short tandem repeat based engraftment test has a high degree of accuracy distinguish the cells of origin from two individuals or in detecting mixtures provided the majority population is present in a proportion of at 1%. Purity of the cell subsets is >90% as determined through protocol validation studies.

TABLE 2
Donor-recipient chimerism testing. Unfractionated
peripheral blood mononuclear cells.

| Days Post-Stem | Chimerism | Recipient Serum |
|---|---|---|
| Cell Infusion | (Donor Cells (%)) | creatinine (mg/dL) |
| 0 | n/a | 1.4 |
| 24 | 26 | 1.9 |
| 54 | 43 | 2.0 |
| 82 | 36 | 1.9 |
| 110 | 32 | 2.1 |
| 138* | 26* | 2.4 |
| 201 | 16 | 1.4 |
| 228** | n/a** | 1.5 |
| 264 | 4 | 1.2 |

*begin tacrolimus weaning.
**complete immunosuppression withdrawal

1. A method of modulating chimerism and/or immunological tolerance in a patient having a pre-existing transplanted organ, the method comprising:
(a) selecting a patient having a transplanted organ, wherein:
the transplanted organ is obtained from an individual who is not genetically identical to the patient; and
the patent is being or has been administrated an immunosuppressive agent that facilitates immunological tolerance of the transplanted organ;
(b) performing a transplantation conditioning regimen on the patient, wherein the transplantation conditioning regimen comprises the administration of rabbit anti-thymocyte globulin and total lymphoid irradiation; and
(c) transfusing the patient with donor hematopoietic stem/progenitor cells and T cells; wherein the donor hematopoietic stem/progenitor and T cells are obtained from the individual from which the transplanted organ was obtained.

2. The method of claim 1, wherein the method comprises administering the patient belumosudil.

3. The method of claim 1, wherein the hematopoietic stem/progenitor cells are obtained by administering one or more mobilization agents to the individual from which the transplanted organ was obtained, wherein the one or more mobilization agents comprise plerixafor, human granulocyte colony-stimulating factor (G-CSF), or a combination thereof.

4. The method of claim 1, wherein the transplanted organ is a kidney, heart, intestine, liver, pancreas or lung.

5. The method of claim 1, further comprising observing a physiological parameter in the patient for a period of time following the transfusing the patient with hematopoietic stem/progenitor cells, wherein the physiological parameter includes at least one of:
detectability of chimerism; estimated glomerular filtration rate (eGFR); and the presence of graft versus host disease.

6. The method of claim 5, wherein the period of time is at least 3, 6, 9 or 12 months.

7. The method of claim 5, further comprising withdrawing the administration of the immunosuppressive agent when the patient exhibits an estimated glomerular filtration rate >30 mL/min and no greater than a 30% change in estimated glomerular filtration rate for at least 3 months.

8. The method of claim 1, wherein the immunosuppressive agent comprises at least one of: an antibody, a calcineurin inhibitor, an antimetabolite, an mTOR inhibitor (mTORi), and a steroid.

9. The method of claim 1, wherein the transplanted organ comprises at least 1, 2, 3, 4, 5 or 6 human leukocyte antigens that are mismatched with human leukocyte antigens present in the patient.

10. The method of claim 9, wherein the HLA mismatches occur at least one of: HLA-A, HLA-B, HLA-C or HLA-DR.

11. The method of claim 1, wherein the patient was transplanted with the organ less than 30 days prior to performing a transplantation conditioning regimen on the patient and then transfusing the patient with hematopoietic stem/progenitor cells and T cells.

12. The method of claim 1, wherein the patient was transplanted with the organ more than 30 days prior to performing a transplantation conditioning regimen on the patient and then transfusing the patient with hematopoietic stem/progenitor cells and T cells.

13. The method of claim 12, wherein the patient was transplanted with the organ more than 1 year, 2 years, 3 years, 4 years or 5 years prior to performing a transplantation conditioning regimen on the patient and then transfusing the patient with hematopoietic stem/progenitor cells and T cells.

14. The method of claim 5, wherein chimerism is detectable in the patient for at least 6, 12, 18 or 24 months following hematopoietic stem/progenitor cells and T cell infusion.

15. The method of claim 14, wherein the patient exhibits a chimerism of at least 10%, 20%, 30% or 40% following the hematopoietic stem/progenitor cells and T cell infusion.

16. The method of claim 5, further comprising initiating weaning the patient off of the immunosuppressive agent when at least one of the following conditions are met: (1) chimerism is detectable for at least 180 days after hematopoietic stem/progenitor cells and T cell transfusion, (2) stable graft function (defined as eGFR >30 mL/min and no greater than sustained 30% change over 3 months from baseline function) without clinical rejection episodes is maintained, and (3) there is no evidence of graft versus host disease (GVHD).

17. The method of claim 16, wherein weaning the patient off of the immunosuppressive agent occurs over a time period of at least 20, 30, 40 or 50 weeks.

18. The method of claim 14, wherein donor leukocytes comprise at least 5%, 10%, 20% or 40% of peripheral blood leukocytes obtained from the patient.
